(19) United States
(12) Patent Application Publication
(10) Pub. No.: US 2007/0167691 A1
(43) Pub. Date: Jul. 19, 2007

(54) Multifunction infant monitoring system

(76) Inventor: Elvir Causevic, New York, NY (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/587,476

(22) PCT Filed: Jan. 28, 2005
(86) PCT No.: PCT/US05/03399

§ 371 Date: Jul. 26, 2006

Related U.S. Application Data
(60) Provisional application No. 60/540,147, filed on Jan. 29, 2004.

(51) Int. Cl. A61B 5/00; A61B 5/04; A61B 5/08

(52) U.S. Cl. 600/301; 600/532; 600/559; 600/322; 600/544; 600/509

(57) ABSTRACT

A medical screening device (100) configured to measure and monitor combinations of blood chemistry, breath gasses, and bioelectrical signals such as evoked auditory signals and EEG signals associated with human auditory testing to facilitate detection of abnormal medical conditions or disorders in human patients by providing a cumulative index representative of at least one detected medical disorder in the human patient.

### CROSS-REFERENCE TO RELATED APPLICATIONS

The present application is related to, and claims priority from, U.S. Provisional Application No. 60/540,147 filed on Jan. 29, 2004, herein incorporated by reference.

### TECHNICAL FIELD

The present invention is related generally to medical screening devices, and in particular, to a medical screening device configured to measure and monitor combinations of blood chemistry, breath gasses, and bioelectrical signals such as evoked auditory signals, EEG signals, and EKG signals associated with infant auditory testing to facilitate detection of abnormal medical conditions in human infants.

### BACKGROUND ART

Hyperbilirubinemia is a serious condition affecting many neonates. Bilirubin is a reddish yellow crystalline pigment C₃₃H₃₆N₄O₆ occurring in bile, blood, urine, and gallstones sometimes in combination with protein and formed by reduction of biliverdin. Bilirubin is produced when red blood cells get old and are broken down by the body. Normally it is processed in the liver and then deposited in the intestine so it can come out in the stool. Bilirubinemia is a condition characterized by elevated bilirubin levels in the bloodstream. The red blood cells of babies have shorter lives than adult red blood cells; bruising at birth may cause a larger number of red cells to be broken down. All of the bilirubin from these cells needs to be processed by the baby's liver. Premature babies do not have fully developed organs. Their livers cannot process bilirubin rapidly. Their intestines may not move much in the first few days especially if they are sick and not being fed. Extremely high levels of bilirubin can be harmful, causing brain damage.

Several tests may be utilized to detect the presence of bilirubinemia or hyperbilirubinemia. These include auditory screening tests, where an detected hearing loss is predictive of bilirubin, a breath gas analysis, where end-tidal carbon-monoxide levels are predictive of bilirubin, and a blood analysis to detect the levels of bilirubin and related blood chemistry components.

For example, the measuring or monitoring of evoked or continuous bioelectric signals in a patient, such as an infant or other human patient who may be incapable of audiometric behavioral responses, is becoming an increasingly common method for initial patient screening or monitoring, and is used in auditory testing programs to identify hearing abnormalities, or in anesthesia and sedation monitoring to determine a patient's state, such as an awareness level.

In auditory screening, the functionality of the outer hair cells of the inner ear can be assessed with measurements of sounds in the external ear canal generated by the inner ear, called otoacoustic emissions (OAE), in response to clicks, called transient evoked OAE (TEOAE), or to two tones, called distortion product OAE (DPOAE).

A TEOAE is generated in response to a transient test signal, usually a sequence of square waves (click). The level of these clicks is typically between 35 dB SPL and 90 dB SPL. In response to these test signals, a normal human ear generates a wide band signal up to 20 ms in duration after each click. The spectrum ST of this response can be compared against the spectrum of ambient noise SA to identify normal or abnormal hearing.

Similarly, a DPOAE is generated in response to the presentation of two simultaneous tonal signals, s1 and s2 [RK1]with associated frequencies f1, and f2, with f2>f2. Typically, the ratio of the frequency of f2 to f1 is selected to be about 1.2, with amplitudes |s1|=65 dB SPL and |s2|=55 db SPL in the ear canal. In response to these signals, a normal human ear generates, among others, a third tonal signal, the DPOAE at frequency 2f1−f2, which can be measured to identify normal or abnormal hearing

Surface electrodes are utilized to detect bioelectric signals in a patient which are generated in response to an auditory stimulus. These bioelectric signals can be used both in auditory screening and in brain activity monitoring during anesthesia or sedation. An auditory evoked potential (AEP) is produced upon presentation of an auditory stimulus or series of stimuli, such as clicks or tone bursts. The AEP can be characterized by three components which refer to the latency of the response with respect to the stimulus; these are referred to as early, middle, and late components.

The early or short latency component of the AEP, the auditory brainstem response (ABR), occurs within the first 15 ms after the presentation of the auditory stimulus and is widely used for clinical evaluation of hearing in infants and other individuals who are unable to effectively communicate whether a sound was detected. In individuals with normal hearing, the ABR generates a characteristic neural waveform. Auditory testing using the ABR typically involves a visual or statistical comparison of a tested individual's waveform to a normal template waveform. Like other evoked potentials, the ABR is recorded from surface electrodes on the scalp. However, the electrodes also record the background noise comprised of unwanted bio-potentials resulting from other neural activity, muscle activity, and unwanted non-physiological sources in the environment.

The middle component of the AEP, the auditory mid-latency response (AMLR), also referred to as the middle latency auditory evoked potential (MLAEP) occurs 15 ms-100 ms after the presentation of the auditory stimulus, and is believed to reflect primary, non-cognitive, cortical processing of auditory stimuli. Lately, the AMLR, or MLAEP, has been of particular interest as a measure of depth of anesthesia, as it is known that the AMLR consists of positive and negative waves that are sensitive to sedatives and anesthetics. In general, increasing the level of sedation or anesthetic increases the latency of these waves, and simultaneously decreases the amplitudes. For monitoring purposes, changes in the AMLR waves are quantified as latency to peak, amplitude, and rate of change, and are sometimes combined in a single index.

The final component of the AEP, the auditory late response (ALR) occurs about 100 ms after the auditory stimulus, and is believed to be especially sensitive to the level of sedation or anesthesia applied to a patient, and exhibits a distinct flattening of the waveform at a relatively light level of sedation or anesthesia, among other features.

It is further known that a 40 Hz auditory signal introduced to a patient's ear can induce an enhanced “steady-state” AEP signal (SSEP) in the patient's nervous system. Conventional signal averaging over a period of time is required to extract this “steady-state” AEP signal from background EEG signals, and adequate responses usually may be obtainable in about 30-40 seconds. The existence of an intact AEP is believed to be a highly specific indicator for the awake state of a patient, and gradual changes in the depth of sedation or anesthesia appear to be reflected by corresponding gradual changes in the AEP.

As with auditory screening procedures, a breath gas analysis can provide a doctor with useful information about a patient's condition and can measure end-tidal carbon-monoxide levels which are predictive of bilirubin. Measurements of the proportions and levels of gases present in a patient's breath are represented by periodic or time-varying gas patterns. Analysis of the gases present in the breath of a patient is commonly utilized as a non-invasive procedure for obtaining a representation of the proportions and levels of gases in the patient's blood. It is known that air in the deep alveolar pockets of a patient's lungs is composed of a mixture of gases which is in close equilibrium with the mixture of gases present in the patient's blood. Accordingly, during a patient's breath cycle, the last portion of an exhalation, i.e. the “end-tidal” portion is believed to provide the most accurate representation of the mixture of gases in the deep alveolar pockets of the lungs, and must be identified and captured for gas analysis.

Finally, blood analysis is a well known procedure for identifying information about the blood chemistry of a patient. Blood analysis may be invasive, wherein a sample of the patient's blood is extracted and analyzed directly, using either chemical reactions or spectroscopic analysis. Alternatively, blood analysis may be non-invasive, wherein spectroscopic analysis is conducted of the patient's blood in-situ, typically by optical spectroscopy of a thin portion of the patient's skin, i.e. the webs of the fingers or finger tips, where light from a light source can pass through the skin and be detected.

It is known that a positive result from two or more different testing methods provides a more accurate test for bilirubinemia or hyperbilirubinemia. Accordingly, it would be highly advantageous to provide a portable, easy to use, medical testing device which is capable of performing a variety of tests, the results-of which may be utilized to predict bilirubinemia or hyperbilirubinemia in a human infant, as well as detect other abnormal conditions which are routinely tested for during infant screening.

### SUMMARY OF THE INVENTION

Briefly stated, the present invention provides a portable medical screening device configured to measure and monitor combinations of blood chemistry, breath gasses, and bioelectrical signals such as evoked auditory signals, EEG signals, and EKG signals associated with human auditory testing to facilitate detection of abnormal medical conditions or disorders in human patients, particularly in infants. The medical screening device includes a portable hand-held enclosure containing a digital signal processor or other suitable logic circuit. The processor is coupled to a memory, and is configured with a computer program capable of conducting multiple testing procedures for a test subject. A display device is coupled to the enclosure, and configured to display patient information, test setup procedures, and test results as directed by the processor. The enclosure further includes a plurality of I/O connection points for various test components linked to the digital signal processor, and to an on-board power supply.

In a first embodiment, a portable medical monitoring system of the present invention is configured for the detection of at least one medical disorder, such as hemolysis, in a human patient. The medical monitoring system consists of an auditory response testing subsystem configured to measure at least one response representative of a first indicator of at least one disorder, such as a hemolysis condition, and at least one non-auditory response testing subsystem. The non-auditory response testing subsystem is configured to measure at least one additional characteristic of the human patient which is representative of the disorder, such as the hemolysis condition. A digital signal processor is operatively coupled to the testing subsystems, and is configured with a computer program operated on command by a user to selectively operate each of the testing subsystems, and to generate a cumulative index representative of a degree of the disorder, responsive to the detected presence of indicators associated with the disorder.

In an alternate embodiment of the present invention, a medical monitoring system for the detection of at least one disorder in a human patient consists of a portable hand-held enclosure within which is disposed a plurality of testing subsystems. Each testing subsystems is configured to measure at least one discrete characteristics of the human patient which is representative of the disorder. Connection points are provided on the enclosure for operatively coupling at least one test instruments to the plurality of testing subsystems, and a self-contained power supply is disposed within the enclosure for operating said plurality of testing systems. Preferably, the testing subsystems include at least an auditory screening subsystem, a breath gas analyzer subsystem, and a blood analysis subsystem.

The foregoing and other objects, features, and advantages of the invention as well as presently preferred embodiments thereof will become more apparent from the reading of the following description in connection with the accompanying drawings.

### BRIEF DESCRIPTION OF THE DRAWINGS

In the accompanying drawings which form part of the specification:

FIG. 1 is a top plan view of one illustrative embodiment of an electrical testing device of the present invention;

FIG. 2 is a view in end elevation of the electrical testing device;

FIG. 3 is a view in end elevation of the electrical testing device end opposite to that shown in FIG. 2;

FIG. 4 is a system block diagram representation of the components of the medical screening device of the present invention; and

FIG. 5 is a software block diagram representing the software components of the medical screening device of the present invention.

Corresponding reference numerals indicate corresponding parts throughout the several figures of the drawings.

### BEST MODES FOR CARRYING OUT THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

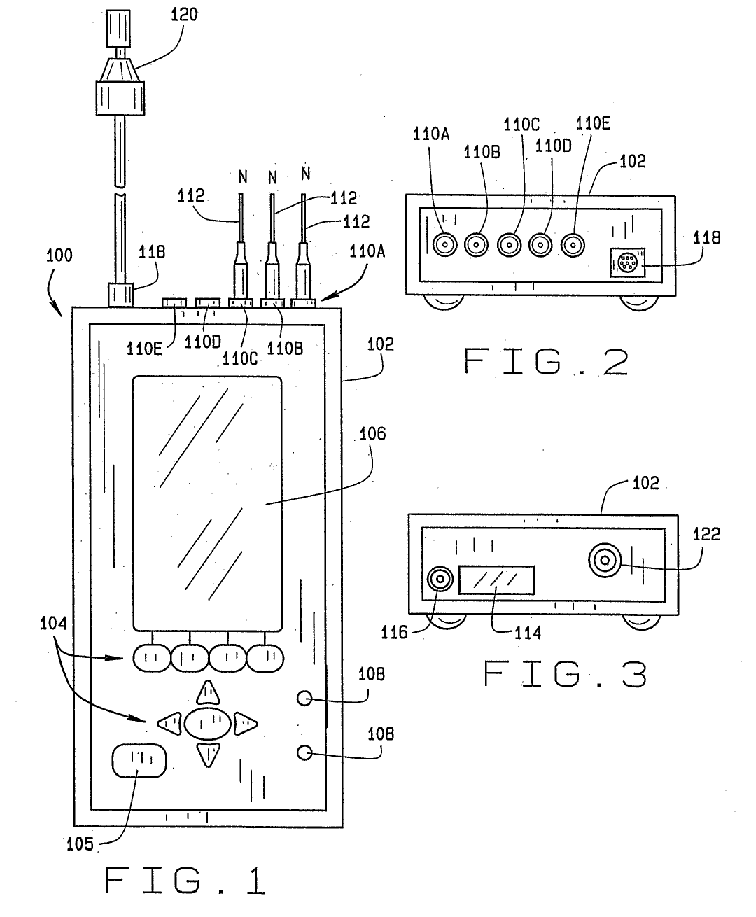

Referring now to FIG. 1 through FIG. 3, reference numeral 100 illustrates one embodiment of the medical screening device of the present invention. The screening device 100 includes an enclosure 102 which can be carried by the user without compromise, and represents a portable hand-held device having full functionality described below. The screening device 100 includes a keyboard 104, which is preferably a membrane switch keyboard incorporating only the minimum keys necessary for operation of the device 100. Preferably, all of the keys on the keyboard 104 are user-programmable, except for the On/Off key 105. A display 106 and at least one function indicator light emitting diode (LED) 108 are also included in the enclosure 102, as well as a set of interfaces 110A-110E for operably coupling a plurality of testing probes, electrodes, gas analyzers, optical probes, or signal output components 112. In the embodiment illustrated, the enclosure 102 further houses an optional infrared communications port 114, and an optional serial communications RS-232 port 116, as well as at least one probe connection 118 suitable for use with an input probe 120, such as an ear probe. A power coupling 122 is provided for connecting the screening device 100 to an external power source, permitting recharging of an internal power source or battery.

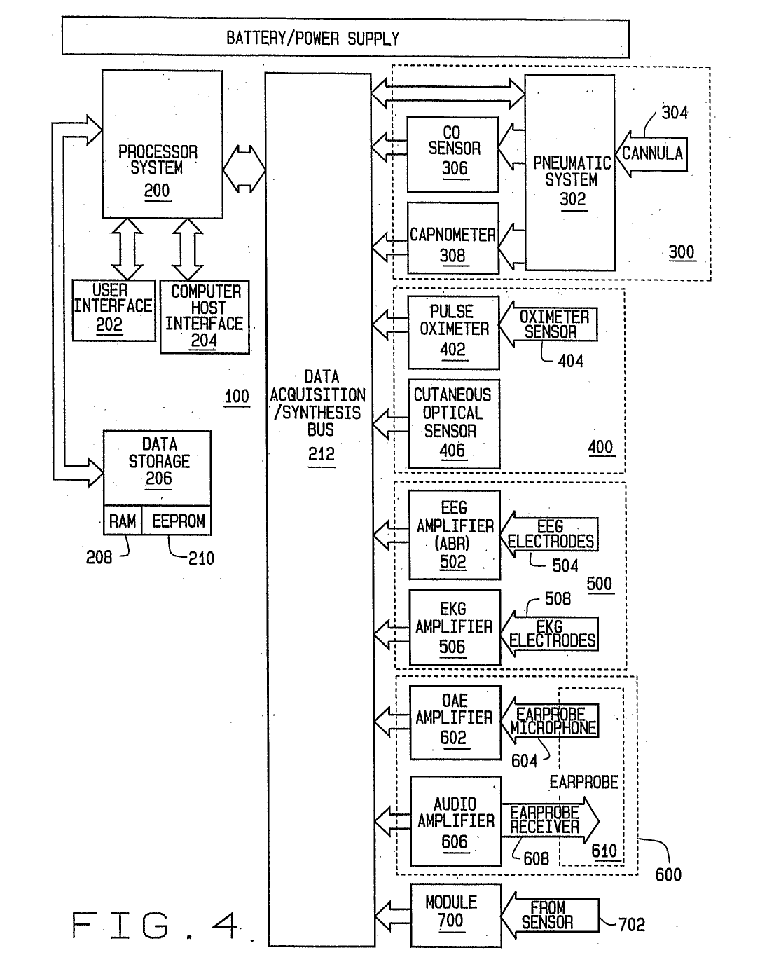

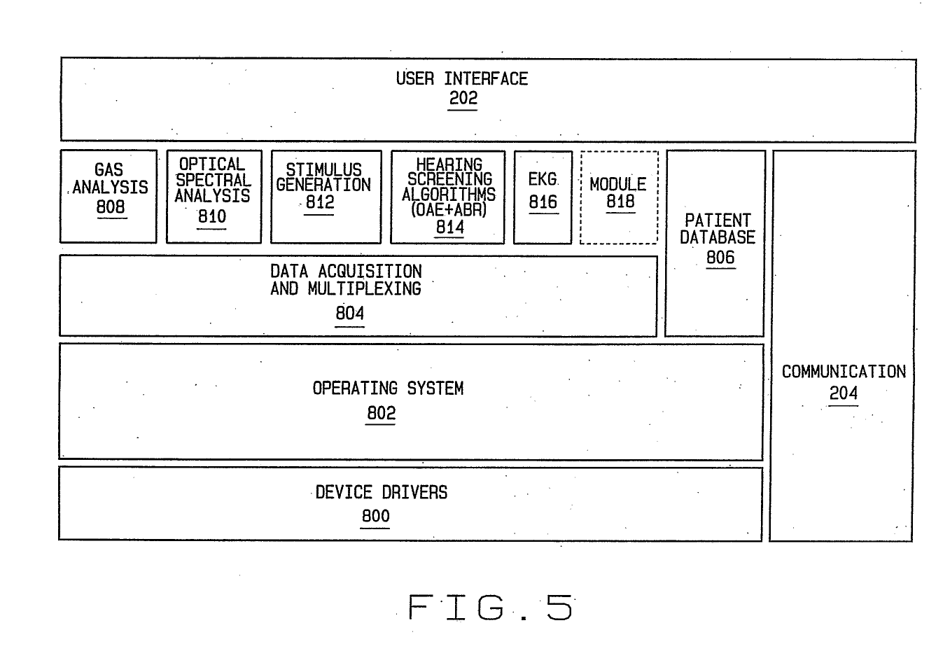

Turning to FIG. 4, a processor system 200 provides the control for the screening device 100. The processor system 200 may include any of a variety of micro-controller or microprocessor circuits, such as a general purpose logic circuit, or a specialized digital signal processor, provided that the processor system 200 is capable of implementing the necessary software instructions to carry out the functions of the screening device 100.

In the preferred embodiment illustrated, all signal processing and data acquisition functions described herein are performed or controlled by the processor system 200. The processor system 200 is further configured to control all input and output functions of the screening device 100, such as graphic or display functions, a user interface 202, a computer host interface 204, patient data storage functions, and other device functionality.

A memory or data storage subsystem 206 is connected to the processor system 200. The memory subsystem 206 includes a random access memory (RAM) 208 for storing intermediate results and holding temporary data, and a flash memory (EPROM or EPROM) 210 for storing non-volatile, electrically programmable variables, patient data, and device configuration information.

Signals for data acquisition and control of external components are conveyed between the processor system 200 and at least one dedicated subsystems or sensor via a data acquisition/data synthesis bus 212. In the embodiment shown in FIG. 4, a breath gas analyzer subsystem 300, a optical blood gas monitor system 400, a bioelectric signal measurement subsystem 500, and an auditory testing subsystem 600 are each operatively coupled to exchange data and instructions through the data acquisition/synthesis bus 212. Those of ordinary skill in the art will recognize that a variety of additional subsystems or modules 700, each configured to receive data from at least one associated sensors 702 may be operatively coupled to the data acquisition/synthesis bus 212.

In one embodiment, the breath gas analyzer subsystem 300 consists of a conventional pneumatic system 302 configured for receiving a patient's breath gas exhalations through a cannula 304 or other suitable breath gas receiver, and for selectively providing samples of the patient's breath gas exhalations to at least one gas monitor such as a carbon monoxide (CO) monitor 306 or a capnometer 308. It is known that the concentration of carbon monoxide gas in a patient's exhaled breath is correlated with the patient's bilirubin production levels, and accordingly, can provide an indicators of an imbalance. For example, a sample of exhaled breath is analyzed to determine the amount of CO in the end-tidal portion of the breath, which can be used as an index of the rate of red blood cell decomposition (hemolysis), which in human neonates can lead to hyperbilirubinemia (jaundice). Alternatively, indicators of other disorders such as lactose malabsorption may be detected through breath gas analysis.

Other breath gas measurements may include, but are not limited to, measurements of the concentrations of oxygen, carbon dioxide, nitrous oxide, and NO₂. Measurements of breath gas concentrations acquired by the gas monitors 306 and 308 are made available to the processor system 200 through the data acquisition/synthesis bus 212. Correspondingly, operation of the pneumatic system 302 is regulated by the processor system 200 through the data acquisition/synthesis bus 212. Those of ordinary skill in the art will recognize that the pneumatic system 302 may include at least one microprocessor or logic circuit which operate independently of the processor system 200 of the screening device 100 in order to carry out functions associated with the pneumatic system 302 and the gas monitors 306, 308, and to provide the measurements of breath gas concentrations to the processor system 200.

In one embodiment, the optical blood gas monitor system 400 consists of a conventional pulse oximeter 402 configured to receive data from an oximeter sensor 404, and a conventional cutaneous optical sensor 406 configured for spectrophotometric analysis of a patient's blood and tissues to determine the presence of, and accumulation of blood analytes, such as, but not limited to, biliruben, hemoglobin, hematocrit, glucose, and lactate. Spectrophotometry works by shining a plain or modulated light at a patient's skin and collecting the reflected light in various spectral bands. The various wavelengths of light present or absent in the reflected light corresponds to the presence and concentration of various analytes in the patient's blood and intervening tissues, permitting the identification of the relative levels of the analytes present in the patient's blood. Pulse oximetery measurements acquired by the pulse oximeter 402, and blood analytes or gas concentrations acquired by the cutaneous optical sensor 406 are made available to the processor system 200 through the data acquisition/synthesis bus 212. Those of ordinary skill in the art will recognize that either the pulse oximeter 402 or the cutaneous optical sensor 406 may include at least one microprocessor or logic circuit which operates independently of the processor system 200 of the screening device 100 in order to carry out functions associated sensors 402, 406, and to provide measured data values to the processor system 200.

In one embodiment, the bioelectric signal measurement subsystem 500 consists of a conventional electro-encephalogram (EEG) measurement system 502 configured to receive bioelectric signals through at least one EEG electrodes 504, and a conventional electrocardiogram (EKG) measurement system 506 configured to receive bioelectric signals through at least one EKG electrodes 508. Output signals from the EEG measurement system 502 and the EKG measurement system 506 are made available to the processor system 200 through the data acquisition/synthesis bus 212. It is known that EEG and EKG signals are predictive or indicative of a variety of medical conditions in a patient. Those of ordinary skill in the art will recognize that either the EEG measurement system 502 or the EKG measurement system 506 may include at least one microprocessor or logic circuit which operates independently of the processor system 200 of the screening device 100 in order to carry out functions associated sensors 502, 506.

In one embodiment, the auditory testing subsystem 600 consists of a conventional otoacoustic emission (OAE) amplifier 602, configured to receive acoustic signals from a microphone 604, and an audio signal amplification system 606, configured to present acoustic signals to a patient's ear through a speaker 608. The microphone 604 and speaker 608 may optionally be combined in a single ear probe 610, configured for insertion into the external ear canal of a patient. Output signals from the OAE amplifier 602 made available to the processor system 200 through the data acquisition/synthesis bus 212. Correspondingly, control signals to the audio signal amplification system 606 are communicated from the processor system 200 to the amplification system 606 through the data acquisition/synthesis bus 212. Those of ordinary skill in the art will recognize that either the OAE amplifier 602 or the audio signal amplification system 606 may include at least one microprocessor or logic circuit which operates independently of the processor system 200 of the screening device 100 in order to carry out functions associated the components 602, 606.

Turning to FIG. 5, computer program code and operating instructions for the processor system 200 are shown. At the basic or physical level, a set of device drivers 800 are configured to handle the necessary data communication and exchanges between the processor system 200 and a variety of interconnected components, such as the user interface 202, computer host interface 204, data store 206, and data acquisition/synthesis bus 212.

Next, the operating system 802 is shown hierarchically above the device drivers, and implements the operating instructions and program code required to direct the device drivers for the exchange of data and control of the various interconnected components. The operating system 802 is further configured to control data acquisition and multiplexing operations 804 on the data acquisition/synthesis bus 212, and to control data retrieval and data storage in the patient database memory structures 806. The operating system 802 further provides a structure in which program modules associated with individual subsystems of the screening device 100 can operate. These modules may include a gas analysis program module 808, an optical spectral analysis module 810, a patient stimulus generator module 812, a hearing screening module 814, a bioelectric signal analysis module 816, and any other optional modules 818 associated with additional subsystems 700 in the screening device 100.

Each program module is configured to receive operator instructions and to display results to an operator through the user interface 820. Correspondingly, each program module is configured for data acquisition, either from an external sensor such as the pneumatic system 302, oximeter sensor 404, EEG or EKG electrodes 504, 508, or earprobe 610 through the data acquisition and multiplexing module 804, or from the patient database 806. Each module 808-818 is configured to operate independently of the other modules present in the screening device 100, and may selectively display obtained results or measurements for an operator. Each module 808-818 may be optionally be configured to utilized results obtained by at least one other module 808-818 to generate one or more combined results, for example, to provide a numerical index representative of a specific condition or disorder in a patient based upon results from different measurements or modules which are representative of characteristics of a particular medical disorder.

For example, measurements obtained by the gas analysis module 808 which are associated with hemolysis may be combined with measurements obtained by the optical spectral analysis module for cutaneous measurements of biliruben accumulation levels to provide an index representative of hemolysis or a biliruben production rate and biliruben accumulation levels which is a powerful indicator of bilirubinemia. Similarly, measurements of other factors from multiple modules may be acquired which are representative of other disorders in the human patient, for example, lactose malabsorption.

Depending upon the particular medical disorders for which the screening device 100 is intended to be utilized, various other optional modules 818 associated with additional subsystems 700 may be added to the screening device 100 to conduct and process specific tests or measurements without departing from the scope of the present invention.

The present invention can be embodied in-part in the form of computer-implemented processes and apparatuses for practicing those processes. The present invention can also be embodied in-part in the form of computer program code containing instructions embodied in tangible media, such as floppy diskettes, CD-ROMs, hard drives, or an other computer readable storage medium, wherein, when the computer program code is loaded into, and executed by, an electronic device such as a computer, micro-processor or logic circuit, the device becomes an apparatus for practicing the invention.

The present invention can also be embodied in-part in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented in a general-purpose microprocessor, the computer program code segments configure the microprocessor to create specific logic circuits.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. A medical monitoring system for the detection of at least one disorder in a human patient, comprising:
a plurality of independently operable modular testing subsystems, said modular testing subsystems including at least
an auditory response testing subsystem, said auditory response testing subsystem configured to measure at least one response of the human patient to an auditory stimulus, said response representative of a first indicator of the at least one disorder; and
at least one non-auditory response testing subsystem, said at least one non-auditory response testing subsystem configured to measure at least one characteristic of the human patient, said at least one characteristic representative of at least one additional indicator of the at least one disorder; and
a processor system operatively coupled to each of said module testing subsystems, said processor system configured with a computer program to selectively operate each of said plurality of module testing subsystems and to generate index value representative of the at least one disorder responsive to a plurality of indicators of the at least one disorder generated by at least two of said modular testing subsystems.

2. The medical monitoring system of claim 1 wherein said at least one non-auditory response testing subsystem includes a breath gas monitoring subsystem.

3. The medical monitoring system of claim 2 wherein said breath gas monitoring subsystem is configured to measure at least a concentration of carbon monoxide exhalations from the human patient, said carbon monoxide concentration an indicator of a hemolysis condition.

4. The medical monitoring system of claim 2 wherein said breath gas monitoring subsystem is configured to measure a concentration of a breath gas exhalations from the human patient selected from a set of breath gases including oxygen, carbon dioxide, nitrous oxide, and NO₂.

5. The medical monitoring system of claim 1 wherein said at least one non-auditory response testing subsystem includes a blood analysis subsystem.

6. The medical monitoring system of claim 5 wherein said blood analysis subsystem is configured to measure at least a presence or absence of at least one chemical compound indicative of a hemolysis condition in a human patient.

7. The medical monitoring system of claim 6 wherein said blood analysis subsystem is further configured for non-invasive optical blood analysis.

8. The medical monitoring system of claim 5 wherein said blood analysis subsystem is configured to measure at least a presence or absence of at least one chemical compound indicative of a lactose malabsorption condition in a human patient.

9. The medical monitoring system of claim 1 further including a portable hand-held enclosure, said processor system, said auditory response testing subsystem, and said at least one non-auditory response testing subsystem disposed within said enclosure;
at least a first connection point on said enclosure for operatively coupling at least a first instrument to said auditory response testing subsystem;
at least a second connection point on said enclosure for operatively coupling at least a second instrument to said at least one non-auditory response testing subsystem; and
a power supply for operating each of said testing subsystems.

10. The medical monitoring system of claim 9 wherein said first and second instruments are selected from a set of instruments including microphones, acoustic emitters, electrodes, gas analyzers, and optical sensors.

11. The medical monitoring system of claim 1 wherein said at least one non-auditory response testing subsystem includes a bioelectric signal measurement subsystem, said bioelectric signal measurement subsystem configured to measure at least one bioelectric signal from a patient.

12. The medical monitoring system of claim 11 wherein said processor system is further configured to evaluate said bioelectric signal to detect at least one predetermined anomaly representative of a medical disorder in a patient.

13. The medical monitoring system of claim 11 wherein said measured bioelectric signal is selected from a set of bioelectric signals including electro-encephalogram signals and electrocardiogram signals.

14. The medical monitoring system of claim 1 wherein said processor system is further configured to selectively display test results from a single testing subsystem.

15. The medical monitoring system of claim 1 wherein said processor system is further configured to selectively display a cumulative index generated from measurements acquired by a plurality of testing subsystems, said cumulative index representative of a medical condition of said human patient.

16. An medical monitoring system for the detection of at least one medical disorder in a human patient, comprising:
a portable hand-held enclosure;
a plurality of testing subsystems disposed within said enclosure, each of said plurality of testing subsystems configured to measure at least one discrete characteristic of the human patient, each of said discrete characteristics representative of the at least one medical disorder;
at least one connection points on said enclosure for operatively coupling at least one instrument to said plurality of testing subsystems;
a power supply for operating said plurality of testing systems; and
wherein said at least one medical disorder is selected from a set of medical disorders including hemolysis, lactose malabsorption, and hyperbilirubinemia.

17. The medical monitoring system of claim 16 wherein said plurality of testing subsystems are selected from a set of testing subsystems including an auditory screening subsystem; a breath gas analyzer subsystem; a blood analysis subsystem; and a bioelectric signal measurement subsystem.

18. The medical monitoring system of claim 17 wherein said blood analysis subsystem is a non-invasive optical blood analysis subsystem.

19. The medical monitoring system of claim 16 wherein said at least one instrument is selected from a set of instruments including microphones, acoustic emitters, electrodes, gas collectors, and optical sensors.

20. The medical monitoring system of claim 16 further including a processor system housed within said enclosure, said processor system having with a computer program configured to selectively operate said plurality of testing subsystems, and to provide results to said user.

21. The medical monitoring system of claim 20 further including a display device mounted to said enclosure, said display device operatively connected to said processor system to display said results.

22. The medical monitoring system of claim 20 wherein said computer program is configured to utilize results from at least two of said testing subsystems to generate a cumulative index value representative of the at least one medical disorder in the human patient.

23. (canceled)

24. A self-contained, portable medical monitoring system for the detection of at least one medical disorder in a human patient, comprising:
a plurality of detection means for acquiring a plurality of different measurements of patient characteristics representative of the at least one medical disorder; and
a processor means coupled to said plurality of detection means, said processor means configured with a plurality of independent program modules for evaluating said plurality of different measurements, each of said plurality of independent program modules associated with one of said plurality of detection means for evaluating measurements acquired by said associated detection means; and
wherein said processor means is further configured to communicate data between at least two of said independent program modules for generating an index value based on at least a portion of said plurality of different measurements, said index value representative of severity of the at least one medical disorder.

25. The self-contained, portable medical monitoring system of claim 24 wherein said processor means is further configured to generate a display of at least one of said plurality of different measurements to a user.
